(19) United States
(12) Patent Application Publication
(10) Pub. No.: US 2023/0337987 A1
(43) Pub. Date: Oct. 26, 2023

(54) DETECTING MOTION ARTIFACTS FROM K-SPACE DATA IN SEGMENTEDMAGNETIC RESONANCE IMAGING

(71) Applicant: The General Hospital Corporation, Boston, MA (US)

(72) Inventors: Stephen Robert Frost, Cambridge, MA (US); Ikbeom Jang, Brighton, MA (US); Jayashree Kalpathy-Cramer, Lynnfield, MA (US)

(21) Appl. No.: 18/305,091

(22) Filed: Apr. 21, 2023

Related U.S. Application Data
(60) Provisional application No. 63/333,373, filed on Apr. 21, 2022.

(51) Int. Cl. A61B 5/00; A61B 5/055; G06T 3/40; G06T 7/246; G06V 10/44; G06V 10/764; G06V 10/82

(52) U.S. Cl. CPC A61B 5/7207; A61B 5/055; G06T 3/40; G06T 7/246; G06V 10/44; G06V 10/764; G06V 10/82; G06T 2207/10088; G06T 2207/20081; G06T 2207/20084

(57) ABSTRACT

Motion artifacts are detected from raw k-space data acquired with a magnetic resonance imaging (“MM”) system. A machine learning model is trained on a training dataset that includes motion-simulated k-space data. The motion-simulated k-space data may be generated by inputting magnetic resonance images to a forward model to convert the images to k-space data while adding motion based on motion parameters. The severity of the simulated motion can be varied, and features of motion artifacts extracted by preprocessing the motion-simulated k-space data. In deployment, the trained machine learning model may be used to detect the presence and/or severity of motion artifacts in k-space data while a subject is being scanned with an MRI scanner.

### CROSS-REFERENCE TO RELATED APPLICATIONS

This application claims the benefit of U.S. Provisional Patent Application Ser. No. 63/333,373, filed on Apr. 21, 2022, and entitled “System for and Method of Detecting Motion Artifacts from k-Space Data in Segmented MRI,” which is herein incorporated by reference in its entirety.

### STATEMENT OF FEDERALLY SPONSORED RESEARCH

This invention was made with government support under EB029641 awarded by the National Institutes of Health. The government has certain rights in the invention.

### BACKGROUND

When a patient undergoing magnetic resonance imaging (“MRI”) makes voluntary, or involuntary, movements during the scan, the resulting images will be corrupted by motion artifacts. Motion artifacts can cause image distortions and degradations that negatively impact clinical diagnosis and the radiology workflow, especially in cases where an imaging recall is required. Detecting motion artifacts while the patient is still in the MRI scanner could improve radiology workflow and reduce costs by enabling efficient corrective action.

### SUMMARY OF THE DISCLOSURE

In some aspects, the present disclosure provides a method for training a neural network to detect motion artifacts in k-space data acquired with a magnetic resonance imaging (MRI) system. The method includes accessing magnetic resonance images and motion parameters with a computer system. Motion-simulated k-space data are generated using a forward model to convert the magnetic resonance images to k-space data while using the motion parameters to apply different degrees of motion to the k-space data. A training dataset is assembled from the motion-simulated k-space data, and a neural network is trained on the training dataset. The resulting trained neural network is then stored for later use.

It is another aspect of the present disclosure to provide a method for detecting motion artifacts in k-space data acquired with an MRI system. The method includes acquiring k-space data from a subject using the MRI system and accessing a machine learning model with a computer system, where the machine learning model has been trained on training data to detect motion artifacts in k-space data. The k-space data are input to the machine learning model, generating motion artifact classification data as an output, where the motion artifact classification data indicate a presence and severity of motion artifacts in the k-space data. The motion artifact classification data may be analyzed with the computer system to control operation of the MRI system.

### BRIEF DESCRIPTION OF THE DRAWINGS

FIG. 1 shows a workflow for an example method of training a machine learning model, such as a deep neural network, to detect motion artifacts in k-space data acquired with an MRI system.

FIG. 2 illustrates a process for generating motion-simulated data. A forward model is used to simulate motion artifacts in two-dimensional multislice data. In the illustrated example, the forward model takes as inputs: a 3D isotropic image, coil sensitivity maps, and head position for the sampling of each k-space segment. The output is multi-channel k-space data where each k-space segment has been sampled from a slice at the supplied head position.

FIGS. 3A and 3B illustrate a process for k_{y} cross-correlation preprocessing of k-space data. The method of normalized cross-correlation between neighboring phase encoding (k_{y}) lines is shown in FIG. 3A. Examples with different levels of simulated motion are shown in FIG. 3B for 2D accelerated multislice T2 FLAIR FSE. In the rightmost plots in FIG. 3B (“ky xcorr mag”), coils are color-coded in the 1D plots and encoded in the y-axis in the 2D plots. The other three columns in FIG. 3B show data from one of the coils.

FIG. 4 is a flowchart setting forth the steps of an example method for generating classified feature data indicating the presence and/or severity of motion artifacts in k-space data by inputting those k-space data to a suitably trained machine learning model.

FIG. 5 is a flowchart setting forth the steps of an example method for training a machine learning model to detect the presence and/or severity of motion artifacts in k-space data.

FIG. 6 is a block diagram of an example system for detecting motion artifacts in k-space data.

FIG. 7 is a block diagram of example components that can implement the system of FIG. 6.

FIG. 8 is a block diagram of an example MRI system that can be implemented in accordance with some examples described in the present disclosure.

### DETAILED DESCRIPTION

Described here are systems and methods for detecting motion artifacts directly from raw k-space data acquired with a magnetic resonance imaging (“MRI”) system. Advantageously, by detecting motion artifacts while the patient is still in the MRI scanner, the overall radiology workflow can be improved by avoiding time-intensive patient recalls. For example, detecting motion artifacts while the patient is still in the scanner could potentially improve workflow by alerting technicians to artifacts during or after a scan acquisition, such that efficient corrective action can be taken. Accordingly, patient costs and operating costs may also be reduced by taking the appropriate corrective action to avoid needing to recall the patient for additional scanning at a different date.

The described systems and methods utilize a supervised learning-based approach to detect motion artifacts directly from raw k-space data. Advantageously, the systems and methods can be used to detect motion artifacts in a variety of imaging applications, including clinically important two-dimensional (“2D”) fast spin echo (“FSE”) multislice scans. By detecting motion artifacts while the subject is still in the MRI scanner, corrective actions can be taken immediately without having to bring the subject back to be rescanned at a later date or time.

A machine learning model, such as a neural network, is trained on training data that include labeled k-space data that have been generated using a motion simulation process that adds subject-motion effects to the data. The motion-simulated data are generated by a framework that takes a magnetic resonance image and associated k-space data as input. Coil sensitivities may be estimated from the k-space data, and subject motion is simulated by applying rigid-body subject motion. The k-space phase-encode lines acquired for the slice of interest may be sampled to form the motion-simulated data. The subject position in the k-space data is transformed to a new position for the next set of k-space lines, and so on, until all the required lines of k-space have been simulated. To avoid relying on imbalanced data (i.e., fewer data with severe motion artifact), varying levels of motion artifact severity can be simulated. Cross-correlation between adjacent phase-encoding lines may be used as features for training.

It is an advantage of the present disclosure that the motion-simulated data may simulate fully 3D subject motion (e.g., head motion, respiration, cardiac motion, etc.) to generate k-space data that would be acquired from 2D excited slices during a segmented k-space acquisition, or other data acquisition scheme as desired. By simulating 3D subject motion, through-slice motion is incorporated into the data generation process, rather than just within-slice translations and rotations about a slice normal.

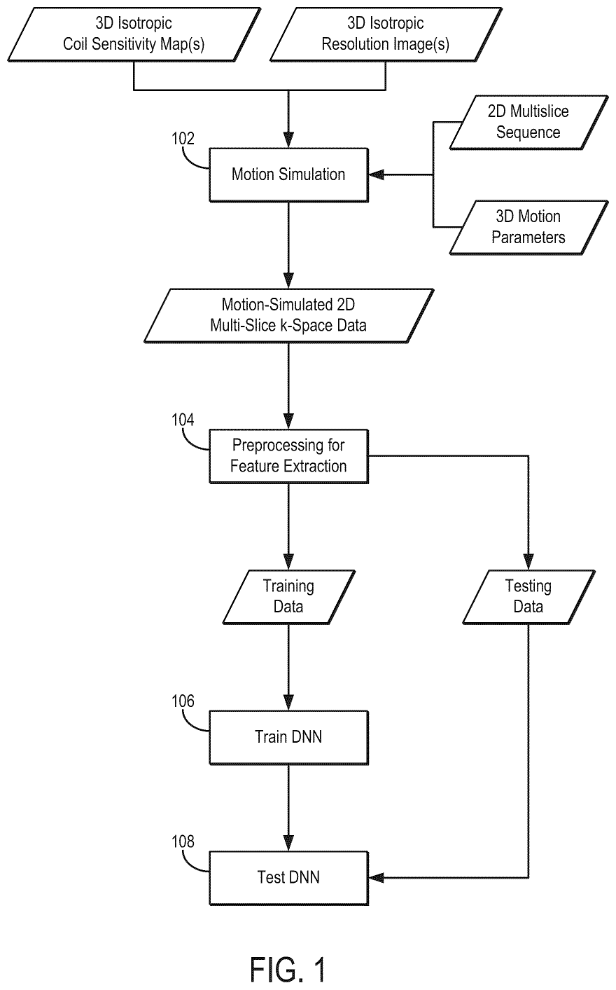

FIG. 1 illustrates an example workflow of a process for training a machine learning model (e.g., a deep neural network (“DNN”) or other suitable machine learning model) to detect motion artifacts in raw k-space data. The process includes collecting data that will be used for training, testing, and validating the machine learning model. For instance, magnetic resonance images and/or their corresponding raw k-space data can be acquired and collected. In the illustrated example, the magnetic resonance images include three-dimensional (“3D”) images with isotropic resolution. The magnetic resonance images and/or raw k-space data will be converted into motion-simulated k-space data that can be used to train, test, and validate the machine learning model.

Additional data that can be collected include coil sensitivity maps and motion parameters to apply to the collected data to simulate patient motion during the data acquisition process. In the illustrated example, the coil sensitivity maps include 3D coil sensitivity maps with isotropic resolution. The motion parameters may be 3D motion parameters, which may include 3D translations (e.g., translation in the x, y, and z direction), 3D rotations (e.g., rotations about the x, y, and z axes), or combinations thereof. Additionally or alternatively, non-rigid deformations of the object can also be simulated for applications in the body (e.g., cardiac imaging applications). Additionally, parameters or other information about a pulse sequence can be collected. These pulse sequence data may be used to convert the collected data into k-space data that are representative of having been acquired by the selected pulse sequence. As a non-limiting example, the pulse sequence data can include information about the k-space sampling provided by the pulse sequence, such as number and distribution of phase encoding lines in k-space, shape and distribution of k-space trajectories, and so on. In the illustrated example, the pulse sequence data include parameters for a two-dimensional (“2D”) multislice pulse sequence, such as a 2D fast spin echo (“FSE”) pulse sequence. For example, the phase sequence data may include a segment phase encoding order for all slices in a multislice acquisition. Pulse sequence parameters for other types of pulse sequences can additionally, or alternatively, be collected and used.

As a non-limiting example, the magnetic resonance images may be images acquired with isotropic resolution using a Cube T2 FLAIR pulse sequence using a multichannel coil array (e.g., a 48-channel coil) with the following imaging parameters: TR=6300 ms, TE=110 ms, FOV=256×230 mm², acquisition matrix=[272, 246], slice thickness=1.0-1.4 mm. In this example, the motion simulation pipeline produced 2D FSE multislice axial T2 FLAIR sequence images (ARC acceleration factor=3, TR=10000 ms, TE=118 ms, FOV=260×260 mm², acquisition matrix=[416, 300], slice thickness=5 mm, slice spacing=1 mm) from the 3D isotropic data.

The collected data are then input to a motion simulator to generate motion-simulated k-space data, as indicated at process block 102. In general, the motion simulator receives the magnetic resonance images (or raw k-space data), adds simulated motion to k-space data according to the motion parameters, and when pulse sequence parameters are supplied converts the resulting motion-simulated k-space data into the appropriate form according to the supplied pulse sequence parameters. For instance, converting the k-space data may include subsampling higher resolution k-space data to match the acquisition provided by the pulse sequence parameters.

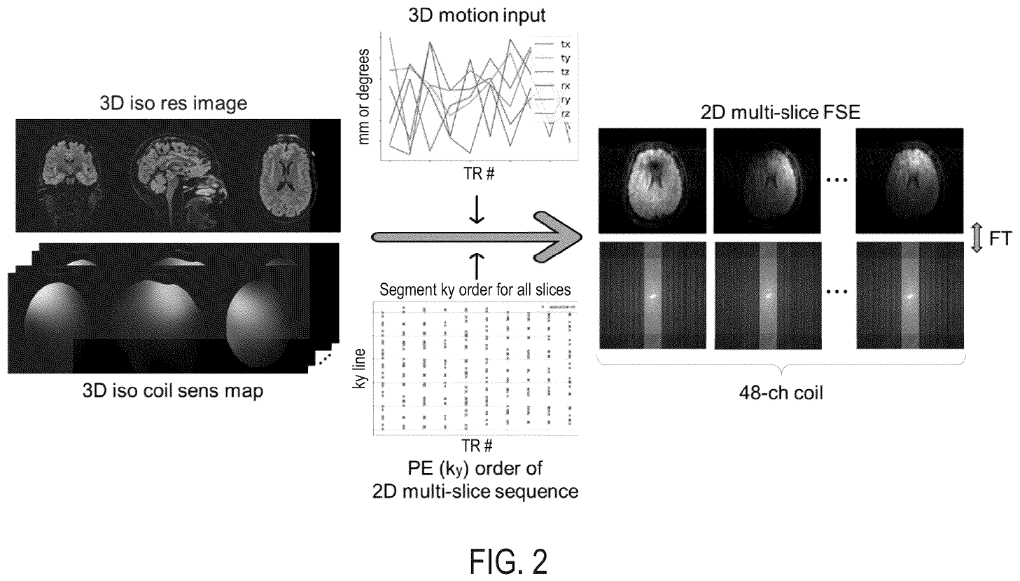

Using knowledge of the k-space sampling provided by the pulse sequence data, a forward model may be used to generate motion-simulated k-space data that simulate motion artifacts. Referring now to FIG. 2, an example process for generating motion-simulated k-space data is shown. In the illustrated example, the motion simulator receives as input: a magnetic resonance image (e.g., a 3D isotropic image), coil sensitivity maps (e.g., coil sensitivity maps estimated using ESPIRiT or other suitable algorithms or techniques), and anatomy and/or slice positions (e.g., head positions) for the sampling of each k-space segment. The output is k-space data where each k-space segment has been sampled from a slice at the supplied position. The forward model enables simulation of both in-plane and through-plane motion. Rigid-body head motion may be used to simulate different levels of motion artifact (e.g., no artifacts, mild artifacts, moderate artifacts, severs artifacts) by controlling the motion parameters. In a non-limiting example, 33,600 k-space datasets (30 studies×28 slices×4 motion severity classes×10 augmentations) were generated, each corresponding to an anatomical slice. The dataset may be split into three datasets at the study level. For example, 60% of the studies may be used for training, 20% for validation, and 20% for testing.

The motion-simulated k-space data are then preprocessed to reduce the dimensionality of the motion-simulated k-space data, as indicated at process block 104. As an example, motion-related features may be extracted from the motion-simulated k-space data, such as by detecting inconsistencies in k-space caused by motion. Based on the assumption that data in neighboring k_{y} phase encoding (“PE”) lines are not very different unless motion occurs, the normalized cross-correlation between adjacent k_{y} lines (which may be referred to as the “k_{y} cross-correlation”) is calculated as:

$$D ( {k}_{y} ) = \frac{1}{2 {K}_{x} + 1} {\sum }_{{k}_{x} = - {K}_{x}}^{{K}_{x}} \frac{{f ( {k}_{x} , {k}_{y} )}^{*} f ( {f}_{x} , {k}_{y} - 1 )}{\vert {f ( {k}_{x} , {k}_{y} )}^{*} f ( {k}_{x} , {k}_{y} - 1 ) \vert} ;$$

where f(kₓ,k_{y}) is 2D k-space and “*” is the complex conjugate. As one example, the magnitude of the cross-correlation, the center of k-space where it is fully sampled (e.g., a self-calibrated region), and data from a reduced number of available coil channels (e.g., 12 coil channels out of 48) can be used. This process may reduce data dimensions from 4 to 2 for each sample. For example, k-space lines with cross-correlation that indicate

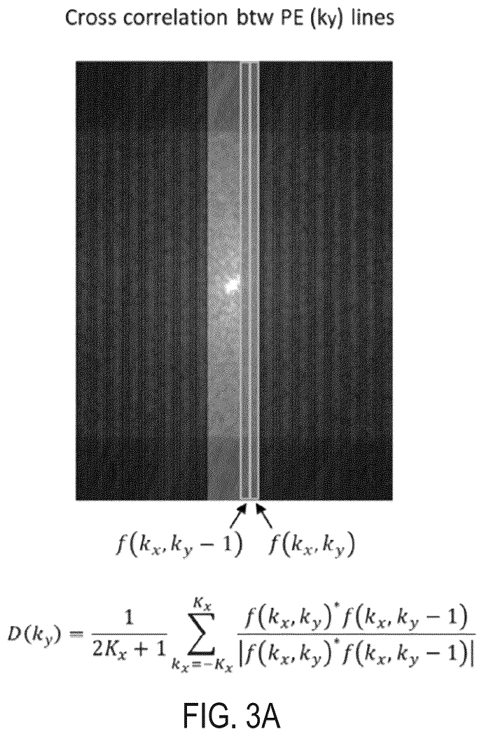

An example workflow of this process is illustrated in FIGS. 3A and 3B. As illustrated in FIG. 3B, the magnitude of the cross-correlation can be analyzed as a feature that indicates the severity of motion and/or motion artifacts. By analyzing the cross-correlation magnitude for k-space lines in the central region of k-space, the severity of motion can be assessed. As shown, those instances with little to no motion have increased cross-correlation, whereas cross-correlation values are reduced as motion becomes more severe.

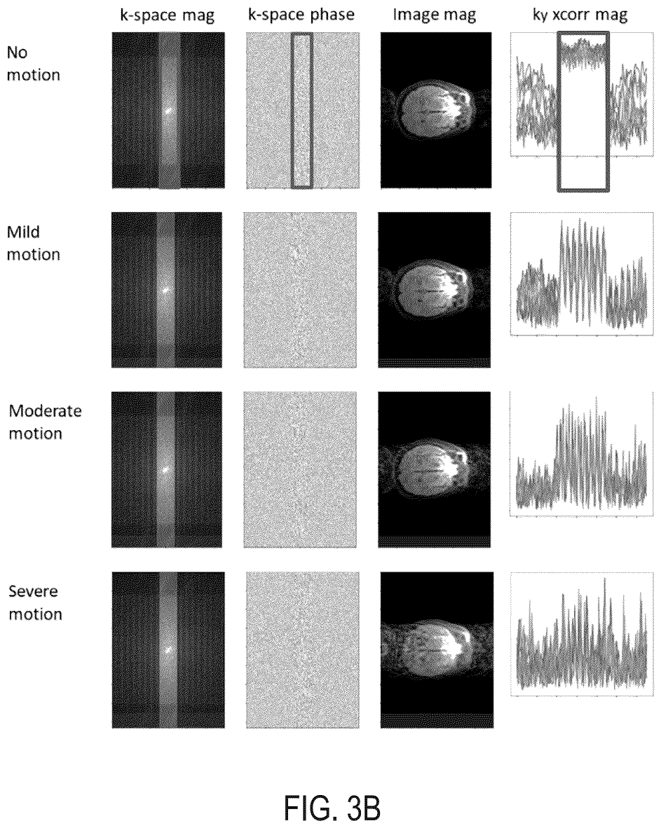

Referring now to FIG. 4, a flowchart is illustrated as setting forth the steps of an example method for generating classified feature data using a suitably trained neural network or other machine learning model. As will be described, the neural network or other machine learning model takes k-space data as input data and generates classified feature data as output data. As an example, the classified feature data can be motion artifact classification data indicative of the presence and/or severity of motion artifacts in the k-space data.

The method includes accessing k-space data with a computer system, as indicated at step 402. Accessing the k-space data may include retrieving such data from a memory or other suitable data storage device or medium. Additionally or alternatively, accessing the k-space data may include acquiring such data with an MRI system and transferring or otherwise communicating the data to the computer system, which may be a part of the MRI system.

A trained neural network (or other suitable machine learning model) is then accessed with the computer system, as indicated at step 404. In general, the neural network is trained, or has been trained, on training data in order to detect the presence of motion artifacts in k-space data and to classify the severity of the detected motion artifacts.

Accessing the trained neural network may include accessing network parameters (e.g., weights, biases, or both) that have been optimized or otherwise estimated by training the neural network on training data. In some instances, retrieving the neural network can also include retrieving, constructing, or otherwise accessing the particular neural network architecture to be implemented. For instance, data pertaining to the layers in the neural network architecture (e.g., number of layers, type of layers, ordering of layers, connections between layers, hyperparameters for layers) may be retrieved, selected, constructed, or otherwise accessed.

An artificial neural network generally includes an input layer, one or more hidden layers (or nodes), and an output layer. Typically, the input layer includes as many nodes as inputs provided to the artificial neural network. The number (and the type) of inputs provided to the artificial neural network may vary based on the particular task for the artificial neural network.

The input layer connects to one or more hidden layers. The number of hidden layers varies and may depend on the particular task for the artificial neural network. Additionally, each hidden layer may have a different number of nodes and may be connected to the next layer differently. For example, each node of the input layer may be connected to each node of the first hidden layer. The connection between each node of the input layer and each node of the first hidden layer may be assigned a weight parameter. Additionally, each node of the neural network may also be assigned a bias value. In some configurations, each node of the first hidden layer may not be connected to each node of the second hidden layer. That is, there may be some nodes of the first hidden layer that are not connected to all of the nodes of the second hidden layer. The connections between the nodes of the first hidden layers and the second hidden layers are each assigned different weight parameters. Each node of the hidden layer is generally associated with an activation function. The activation function defines how the hidden layer is to process the input received from the input layer or from a previous input or hidden layer. These activation functions may vary and be based on the type of task associated with the artificial neural network and also on the specific type of hidden layer implemented.

Each hidden layer may perform a different function. For example, some hidden layers can be convolutional hidden layers which can, in some instances, reduce the dimensionality of the inputs. Other hidden layers can perform statistical functions such as max pooling, which may reduce a group of inputs to the maximum value; an averaging layer; batch normalization; and other such functions. In some of the hidden layers each node is connected to each node of the next hidden layer, which may be referred to then as dense layers. Some neural networks including more than, for example, three hidden layers may be considered deep neural networks.

The last hidden layer in the artificial neural network is connected to the output layer. Similar to the input layer, the output layer typically has the same number of nodes as the possible outputs. In an example in which the artificial neural network detects and/or classifies motion artifacts in k-space data, the output layer may include, for example, a number of different nodes, where each different node corresponds to a different class of motion artifact severity. A first node may indicate no motion artifacts in the k-space data, a second node may indicate mild motion artifacts in the k-space data, a third node may indicate moderate motion artifacts in the k-space data, and a fourth node may indicate severe motion artifacts in the k-space data. Additionally or alternatively, the outputs may include a confidence score and/or probability for each classification.

The k-space data are then input to the one or more trained neural networks, generating output as classified feature data, as indicated at step 406. For example, the classified feature data may include motion artifact classification data. Accordingly, the classified feature data may indicate the probability for the presence of motion artifacts in the k-space data and/or the probability of a particular classification (i.e., the probability that the k-space data include patterns, features, or characteristics indicative of detecting, differentiating, and/or determining the severity of motion artifacts in the k-space data). Additionally or alternatively, the classified feature data may classify the k-space data as indicating a particular severity of motion artifacts. In these instances, the classified feature data can differentiate between different degrees of motion artifact.

The classified feature data generated by inputting the k-space data to the trained neural network(s) can then be displayed to a user, stored for later use or further processing, or both, as indicated at step 408. For example, the classified feature data may include motion artifact classification data indicating the presence and/or severity of motion artifacts in the acquired k-space data. This information can be presented to a user (e.g., a radiology technician operating the MRI scanner) to alert them to the presence of motion artifacts in the k-space data (and the severity thereof), either while the k-space data are being acquired or after a scan prescription has been completed, but while the subject is still in the MRI scanner. In this way, a new scan can be performed before the subject has been removed from the scanner. By providing this feedback to the MRI scanner operator, the overall radiology workflow can be improved and operating costs reduced by avoiding having to recall the patient for additional scanning after they have left the facility.

Additionally or alternatively, when the motion artifacts are detected while the subject is being scanned, the scan can be stopped and restarted to acquire new k-space data that are not corrupted by subject motion. In some examples, the motion artifact classification data may be processed by the computer system to automatically control the operation of the MRI scanner, such as by stopping the scan when motion artifacts are detected at a threshold level of severity (e.g., when moderate or severe motion artifacts are detected).

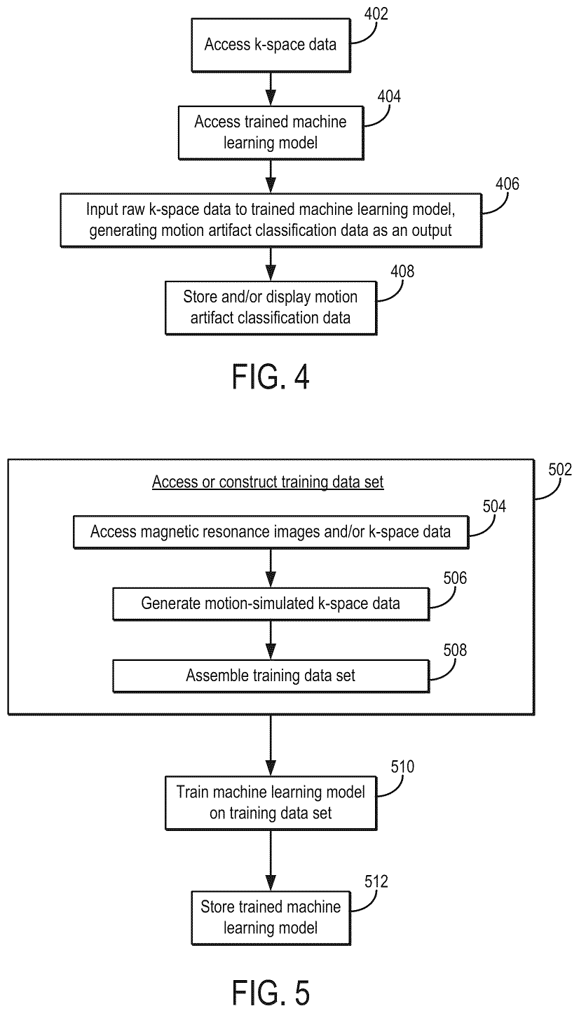

Referring now to FIG. 5, a flowchart is illustrated as setting forth the steps of an example method for training one or more neural networks (or other suitable machine learning models) on training data, such that the one or more neural networks are trained to receive k-space data as input data in order to generate classified feature data as output data. The classified feature data may include motion artifact classification data that are indicative the presence and severity of motion artifacts in the k-space data.

In general, the neural network(s) can implement any number of different neural network architectures. For instance, the neural network(s) could implement a convolutional neural network, a residual neural network, or the like. Alternatively, the neural network(s) could be replaced with other suitable machine learning or artificial intelligence algorithms, such as those based on supervised learning, unsupervised learning, deep learning, ensemble learning, dimensionality reduction, and so on.

As one non-limiting example, the neural network may be a convolutional neural network (“CNN”) or other deep neural network (“DNN”). The convolutional neural network may have any suitable form of architecture, such as a ResNet architecture. In one non-limiting example, the CNN may have a ResNet-18 architecture that has been modified to have a single channel, and in which the fully connected layer was modified to output 4 values corresponding to the four motion artifact severity classifications: no artifact, mild artifact, moderate artifact, and severe artifact.

The method includes accessing training data with a computer system, as indicated at process block 502. Accessing the training data may include retrieving such data from a memory or other suitable data storage device or medium. Alternatively, accessing the training data may include generating such data with the computer system and transferring or otherwise communicating the data to the computer system. For instance, training data may be generated using the workflow described above, in which motion-simulated k-space date are generated and processed to create training data. In these instances, accessing the training data may include accessing magnetic resonance images and/or k-space data (substep 504), generating motion-simulated k-space data therefrom (substep 506), and assembling the training data from the motion-simulated k-space data (substep 508).

In general, the training data include motion-corrupted k-space data. As described above, the motion-corrupted k-space data may be motion-simulated k-space data, in which simulated motion effects have been added to k-space data. Additionally or alternatively, the motion-corrupted k-space data may include k-space data acquired from subjects who were moving when the data were acquired, such that the resulting k-space data are corrupted by motion artifacts. The training data may include motion-corrupted k-space data that have been labeled (e.g., labeled as containing motion artifacts at different levels of severity, such as no artifacts, mild artifacts, moderate artifacts, and severe artifacts).

As noted above, the method can include assembling training data from motion-corrupted k-space using a computer system. This step may include assembling the motion-corrupted k-space into an appropriate data structure on which the neural network or other machine learning model can be trained. Assembling the training data may include generating labeled data and including the labeled data in the training data. Labeled data may include motion-corrupted k-space data that have been labeled as belonging to, or otherwise being associated with, one or more different classifications or categories. For instance, labeled data may include motion-corrupted k-space data that have been labeled as containing no motion artifacts, mild motion artifacts, moderate motion artifacts, or severe motion artifacts.

As described above, assembling the training data may include receiving or otherwise accessing acquired k-space and/or magnetic resonance images and generating motion-simulated k-space data therefrom. For example, magnetic resonance images can be accessed by the computer system. Additionally, coil sensitivity maps associated with the MRI system used to acquire the magnetic resonance images are also accessed by the computer system. In some instances, accessing the coil sensitivity maps may include estimating the coil sensitivity maps from relevant data.

One or more neural networks (or other suitable machine learning models) are trained on the training data, as indicated at step 510. In general, the neural network can be trained by optimizing network parameters (e.g., weights, biases, or both) based on minimizing a loss function. As one non-limiting example, the loss function may be a mean squared error loss function.

Training a neural network may include initializing the neural network, such as by computing, estimating, or otherwise selecting initial network parameters (e.g., weights, biases, or both). During training, an artificial neural network receives the inputs for a training example and generates an output using the bias for each node, and the connections between each node and the corresponding weights. For instance, training data can be input to the initialized neural network, generating output as classified feature data indicating the presence and/or severity of motion artifacts. The artificial neural network then compares the generated output with the actual output of the training example in order to evaluate the quality of the classified feature data. For instance, the classified feature data can be passed to a loss function to compute an error. The current neural network can then be updated based on the calculated error (e.g., using backpropagation methods based on the calculated error). For instance, the current neural network can be updated by updating the network parameters (e.g., weights, biases, or both) in order to minimize the loss according to the loss function. The training continues until a training condition is met. The training condition may correspond to, for example, a predetermined number of training examples being used, a minimum accuracy threshold being reached during training and validation, a predetermined number of validation iterations being completed, and the like. When the training condition has been met (e.g., by determining whether an error threshold or other stopping criterion has been satisfied), the current neural network and its associated network parameters represent the trained neural network. Different types of training processes can be used to adjust the bias values and the weights of the node connections based on the training examples. The training processes may include, for example, gradient descent, Newton's method, conjugate gradient, quasi-Newton, Levenberg-Marquardt, among others.

The artificial neural network can be constructed or otherwise trained based on training data using one or more different learning techniques, such as supervised learning, unsupervised learning, reinforcement learning, ensemble learning, active learning, transfer learning, or other suitable learning techniques for neural networks. As an example, supervised learning involves presenting a computer system with example inputs and their actual outputs (e.g., categorizations). In these instances, the artificial neural network is configured to learn a general rule or model that maps the inputs to the outputs based on the provided example input-output pairs.

The one or more trained neural networks are then stored for later use, as indicated at step 512. Storing the neural network(s) may include storing network parameters (e.g., weights, biases, or both), which have been computed or otherwise estimated by training the neural network(s) on the training data. Storing the trained neural network(s) may also include storing the particular neural network architecture to be implemented. For instance, data pertaining to the layers in the neural network architecture (e.g., number of layers, type of layers, ordering of layers, connections between layers, hyperparameters for layers) may be stored.

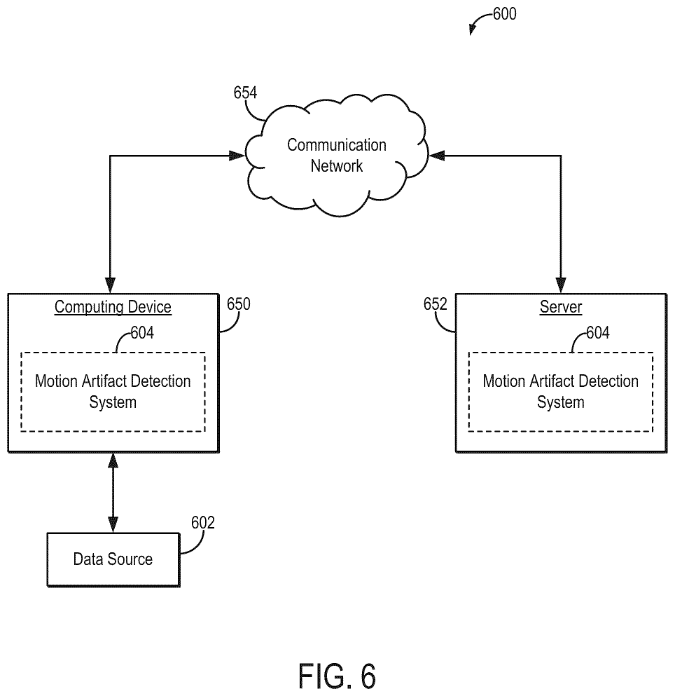

FIG. 6 shows an example of a system 600 for detecting the presence of motion artifacts in raw k-space data in accordance with some embodiments of the systems and methods described in the present disclosure. As shown in FIG. 6, a computing device 650 can receive one or more types of data (e.g., magnetic resonance image, k-space data, coil sensitivity data, motion parameters, k-space sampling or other pulse sequence data) from data source 602. In some embodiments, computing device 650 can execute at least a portion of a motion artifact detection system 604 to detect the presence and/or severity of motion artifacts from k-space data received from the data source 602.

Additionally or alternatively, in some embodiments, the computing device 650 can communicate information about data received from the data source 602 to a server 652 over a communication network 654, which can execute at least a portion of the motion artifact detection system 604. In such embodiments, the server 652 can return information to the computing device 650 (and/or any other suitable computing device) indicative of an output of the motion artifact detection system 604.

In some embodiments, computing device 650 and/or server 652 can be any suitable computing device or combination of devices, such as a desktop computer, a laptop computer, a smartphone, a tablet computer, a wearable computer, a server computer, a virtual machine being executed by a physical computing device, and so on. The computing device 650 and/or server 652 can also reconstruct images from the data.

In some embodiments, data source 602 can be any suitable source of data (e.g., measurement data, images reconstructed from measurement data, processed image data), such as an MRI system, another computing device (e.g., a server storing measurement data, images reconstructed from measurement data, processed image data), and so on. In some embodiments, data source 602 can be local to computing device 650. For example, data source 602 can be incorporated with computing device 650 (e.g., computing device 650 can be configured as part of a device for measuring, recording, estimating, acquiring, or otherwise collecting or storing data). As another example, data source 602 can be connected to computing device 650 by a cable, a direct wireless link, and so on. Additionally or alternatively, in some embodiments, data source 602 can be located locally and/or remotely from computing device 650, and can communicate data to computing device 650 (and/or server 652) via a communication network (e.g., communication network 654).

In some embodiments, communication network 654 can be any suitable communication network or combination of communication networks. For example, communication network 654 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 654 can be a local area network, a wide area network, a public network (e.g., the Internet), a private or semi-private network (e.g., a corporate or university intranet), any other suitable type of network, or any suitable combination of networks. Communications links shown in FIG. 6 can each be any suitable communications link or combination of communications links, such as wired links, fiber optic links, Wi-Fi links, Bluetooth links, cellular links, and so on.

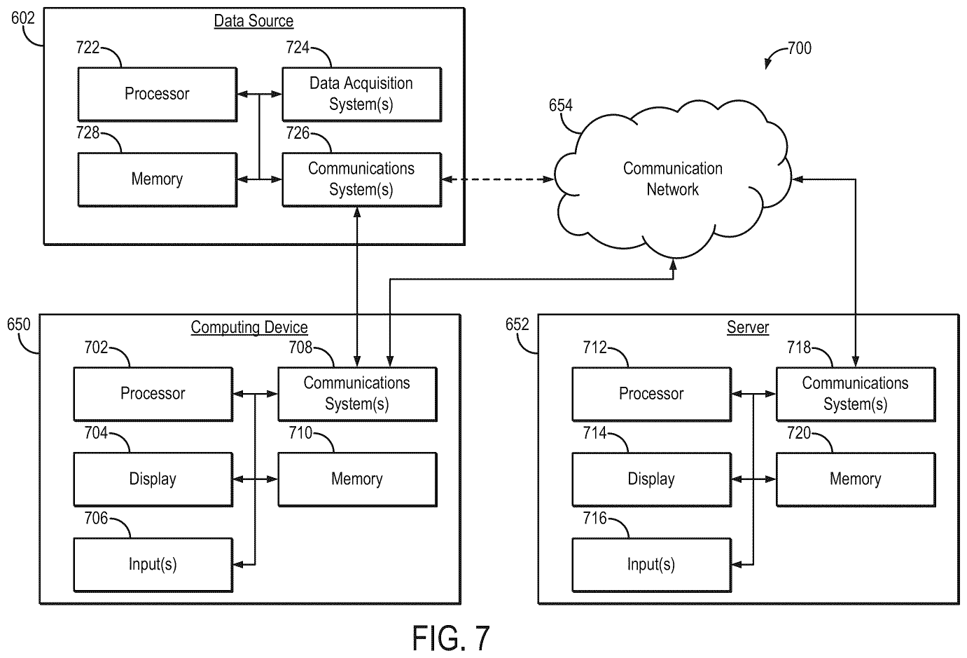

Referring now to FIG. 7, an example of hardware 700 that can be used to implement data source 602, computing device 650, and server 652 in accordance with some embodiments of the systems and methods described in the present disclosure is shown.

As shown in FIG. 7, in some embodiments, computing device 650 can include a processor 702, a display 704, one or more inputs 706, one or more communication systems 708, and/or memory 710. In some embodiments, processor 702 can be any suitable hardware processor or combination of processors, such as a central processing unit (“CPU”), a graphics processing unit (“GPU”), and so on. In some embodiments, display 704 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 706 can include any suitable input devices and/or sensors that can be used to receive user input, such as a keyboard, a mouse, a touchscreen, a microphone, and so on.

In some embodiments, communications systems 708 can include any suitable hardware, firmware, and/or software for communicating information over communication network 654 and/or any other suitable communication networks. For example, communications systems 708 can include one or more transceivers, one or more communication chips and/or chip sets, and so on. In a more particular example, communications systems 708 can include hardware, firmware, and/or software that can be used to establish a Wi-Fi connection, a Bluetooth connection, a cellular connection, an Ethernet connection, and so on.

In some embodiments, memory 710 can include any suitable storage device or devices that can be used to store instructions, values, data, or the like, that can be used, for example, by processor 702 to present content using display 704, to communicate with server 652 via communications system(s) 708, and so on. Memory 710 can include any suitable volatile memory, non-volatile memory, storage, or any suitable combination thereof. For example, memory 710 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 710 can have encoded thereon, or otherwise stored therein, a computer program for controlling operation of computing device 650. In such embodiments, processor 702 can execute at least a portion of the computer program to present content (e.g., images, user interfaces, graphics, tables), receive content from server 652, transmit information to server 652, and so on. For example, the processor 702 and the memory 710 can be configured to perform the methods described herein (e.g., the workflow of FIG. 1, the process illustrated in FIG. 2, the process illustrated in FIGS. 3A and 3B, method of FIG. 4, the method of FIG. 5).

In some embodiments, server 652 can include a processor 712, a display 714, one or more inputs 716, one or more communications systems 718, and/or memory 720. In some embodiments, processor 712 can be any suitable hardware processor or combination of processors, such as a CPU, a GPU, and so on. In some embodiments, display 714 can include any suitable display devices, such as an LCD screen, LED display, OLED display, electrophoretic display, a computer monitor, a touchscreen, a television, and so on. In some embodiments, inputs 716 can include any suitable input devices and/or sensors that can be used to receive user input, such as a keyboard, a mouse, a touchscreen, a microphone, and so on.

In some embodiments, communications systems 718 can include any suitable hardware, firmware, and/or software for communicating information over communication network 654 and/or any other suitable communication networks. For example, communications systems 718 can include one or more transceivers, one or more communication chips and/or chip sets, and so on. In a more particular example, communications systems 718 can include hardware, firmware, and/or software that can be used to establish a Wi-Fi connection, a Bluetooth connection, a cellular connection, an Ethernet connection, and so on.

In some embodiments, memory 720 can include any suitable storage device or devices that can be used to store instructions, values, data, or the like, that can be used, for example, by processor 712 to present content using display 714, to communicate with one or more computing devices 650, and so on. Memory 720 can include any suitable volatile memory, non-volatile memory, storage, or any suitable combination thereof. For example, memory 720 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 720 can have encoded thereon a server program for controlling operation of server 652. In such embodiments, processor 712 can execute at least a portion of the server program to transmit information and/or content (e.g., data, images, a user interface) to one or more computing devices 650, receive information and/or content from one or more computing devices 650, receive instructions from one or more devices (e.g., a personal computer, a laptop computer, a tablet computer, a smartphone), and so on.

In some embodiments, the server 652 is configured to perform the methods described in the present disclosure. For example, the processor 712 and memory 720 can be configured to perform the methods described herein (e.g., the workflow of FIG. 1, the process illustrated in FIG. 2, the process illustrated in FIGS. 3A and 3B, method of FIG. 4, the method of FIG. 5).

In some embodiments, data source 602 can include a processor 722, one or more data acquisition systems 724, one or more communications systems 726, and/or memory 728. In some embodiments, processor 722 can be any suitable hardware processor or combination of processors, such as a CPU, a GPU, and so on. In some embodiments, the one or more data acquisition systems 724 are generally configured to acquire data, images, or both, and can include an MRI system. Additionally or alternatively, in some embodiments, the one or more data acquisition systems 724 can include any suitable hardware, firmware, and/or software for coupling to and/or controlling operations of an MRI system. In some embodiments, one or more portions of the data acquisition system(s) 724 can be removable and/or replaceable.

Note that, although not shown, data source 602 can include any suitable inputs and/or outputs. For example, data source 602 can include input devices and/or sensors that can be used to receive user input, such as a keyboard, a mouse, a touchscreen, a microphone, a trackpad, a trackball, and so on. As another example, data source 602 can include any suitable display devices, such as an LCD screen, an LED display, an OLED display, an electrophoretic display, a computer monitor, a touchscreen, a television, etc., one or more speakers, and so on.

In some embodiments, communications systems 726 can include any suitable hardware, firmware, and/or software for communicating information to computing device 650 (and, in some embodiments, over communication network 654 and/or any other suitable communication networks). For example, communications systems 726 can include one or more transceivers, one or more communication chips and/or chip sets, and so on. In a more particular example, communications systems 726 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 728 can include any suitable storage device or devices that can be used to store instructions, values, data, or the like, that can be used, for example, by processor 722 to control the one or more data acquisition systems 724, and/or receive data from the one or more data acquisition systems 724; to generate images from data; present content (e.g., data, images, a user interface) using a display; communicate with one or more computing devices 650; and so on. Memory 728 can include any suitable volatile memory, non-volatile memory, storage, or any suitable combination thereof. For example, memory 728 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 728 can have encoded thereon, or otherwise stored therein, a program for controlling operation of data source 602. In such embodiments, processor 722 can execute at least a portion of the program to generate images, transmit information and/or content (e.g., data, images, a user interface) to one or more computing devices 650, receive information and/or content from one or more computing devices 650, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

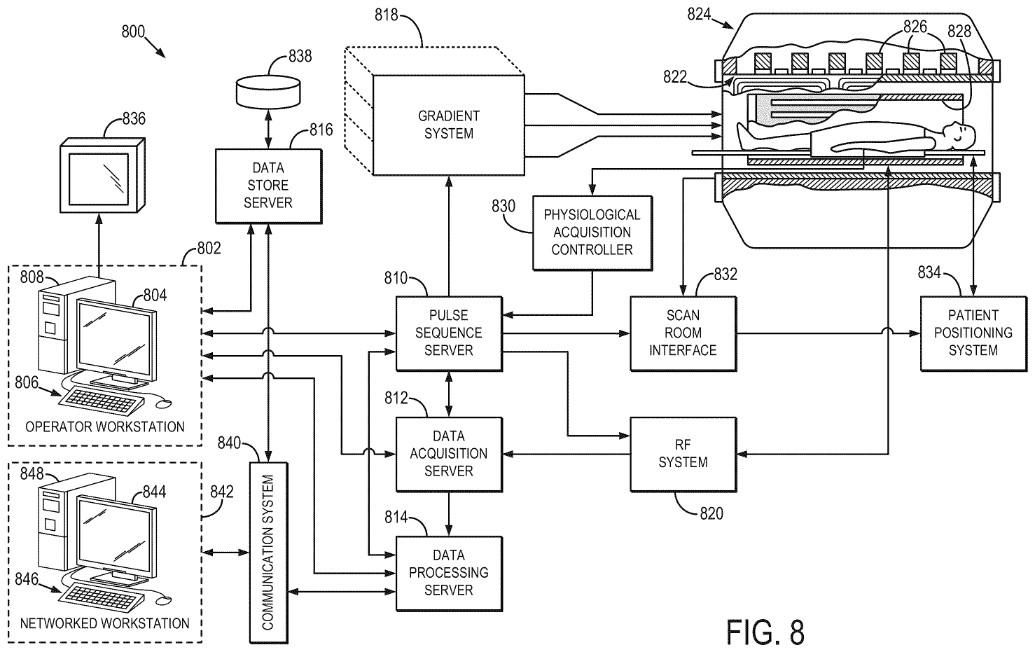

Referring particularly now to FIG. 8, an example of an MRI system 800 that can implement the methods described here is illustrated. The MRI system 800 includes an operator workstation 802 that may include a display 804, one or more input devices 806 (e.g., a keyboard, a mouse), and a processor 808. The processor 808 may include a commercially available programmable machine running a commercially available operating system. The operator workstation 802 provides an operator interface that facilitates entering scan parameters into the MRI system 800. The operator workstation 802 may be coupled to different servers, including, for example, a pulse sequence server 810, a data acquisition server 812, a data processing server 814, and a data store server 816. The operator workstation 802 and the servers 810, 812, 814, and 816 may be connected via a communication system 840, which may include wired or wireless network connections.

The pulse sequence server 810 functions in response to instructions provided by the operator workstation 802 to operate a gradient system 818 and a radiofrequency (“RF”) system 820. Gradient waveforms for performing a prescribed scan are produced and applied to the gradient system 818, which then excites gradient coils in an assembly 822 to produce the magnetic field gradients Gₓ, G_{y}, and G_{z} that are used for spatially encoding magnetic resonance signals. The gradient coil assembly 822 forms part of a magnet assembly 824 that includes a polarizing magnet 826 and a whole-body RF coil 828.

RF waveforms are applied by the RF system 820 to the RF coil 828, or a separate local coil to perform the prescribed magnetic resonance pulse sequence. Responsive magnetic resonance signals detected by the RF coil 828, or a separate local coil, are received by the RF system 820. The responsive magnetic resonance signals may be amplified, demodulated, filtered, and digitized under direction of commands produced by the pulse sequence server 810. The RF system 820 includes an RF transmitter for producing a wide variety of RF pulses used in MRI pulse sequences. The RF transmitter is responsive to the prescribed scan and direction from the pulse sequence server 810 to produce RF pulses of the desired frequency, phase, and pulse amplitude waveform. The generated RF pulses may be applied to the whole-body RF coil 828 or to one or more local coils or coil arrays.

The RF system 820 also includes one or more RF receiver channels. An RF receiver channel includes an RF preamplifier that amplifies the magnetic resonance signal received by the coil 828 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

M=\sqrt{I²+Q²};
and the phase of the received magnetic resonance signal may also be determined according to the following relationship:

$$φ = {tan}^{- 1} ( \frac{Q}{I} ) .$$

The pulse sequence server 810 may receive patient data from a physiological acquisition controller 830. By way of example, the physiological acquisition controller 830 may receive signals from a number of different sensors connected to the patient, including electrocardiograph (“ECG”) signals from electrodes, or respiratory signals from a respiratory bellows or other respiratory monitoring devices. These signals may be used by the pulse sequence server 810 to synchronize, or “gate,” the performance of the scan with the subject's heart beat or respiration.

The pulse sequence server 810 may also connect to a scan room interface circuit 832 that receives signals from various sensors associated with the condition of the patient and the magnet system. Through the scan room interface circuit 832, a patient positioning system 834 can receive commands to move the patient to desired positions during the scan.

The digitized magnetic resonance signal samples produced by the RF system 820 are received by the data acquisition server 812. The data acquisition server 812 operates in response to instructions downloaded from the operator workstation 802 to receive the real-time magnetic resonance data and provide buffer storage, so that data is not lost by data overrun. In some scans, the data acquisition server 812 passes the acquired magnetic resonance data to the data processor server 814. In scans that require information derived from acquired magnetic resonance data to control the further performance of the scan, the data acquisition server 812 may be programmed to produce such information and convey it to the pulse sequence server 810. For example, during pre-scans, magnetic resonance data may be acquired and used to calibrate the pulse sequence performed by the pulse sequence server 810. As another example, navigator signals may be acquired and used to adjust the operating parameters of the RF system 820 or the gradient system 818, or to control the view order in which k-space is sampled. In still another example, the data acquisition server 812 may also process magnetic resonance signals used to detect the arrival of a contrast agent in a magnetic resonance angiography (“MRA”) scan. For example, the data acquisition server 812 may acquire magnetic resonance data and processes it in real-time to produce information that is used to control the scan.

The data processing server 814 receives magnetic resonance data from the data acquisition server 812 and processes the magnetic resonance data in accordance with instructions provided by the operator workstation 802. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 814 are conveyed back to the operator workstation 802 for storage. Real-time images may be stored in a data base memory cache, from which they may be output to operator display 802 or a display 836. Batch mode images or selected real time images may be stored in a host database on disc storage 838. When such images have been reconstructed and transferred to storage, the data processing server 814 may notify the data store server 816 on the operator workstation 802. The operator workstation 802 may be used by an operator to archive the images, produce films, or send the images via a network to other facilities.

The MRI system 800 may also include one or more networked workstations 842. For example, a networked workstation 842 may include a display 844, one or more input devices 846 (e.g., a keyboard, a mouse), and a processor 848. The networked workstation 842 may be located within the same facility as the operator workstation 802, or in a different facility, such as a different healthcare institution or clinic.

The networked workstation 842 may gain remote access to the data processing server 814 or data store server 816 via the communication system 840. Accordingly, multiple networked workstations 842 may have access to the data processing server 814 and the data store server 816. In this manner, magnetic resonance data, reconstructed images, or other data may be exchanged between the data processing server 814 or the data store server 816 and the networked workstations 842, such that the data or images may be remotely processed by a networked workstation 842.

The present disclosure has described one or more preferred embodiments, and it should be appreciated that many equivalents, alternatives, variations, and modifications, aside from those expressly stated, are possible and within the scope of the invention.

1. A method for training a neural network to detect motion artifacts in k-space data acquired with a magnetic resonance imaging (MRI) system, the method comprising:
(a) accessing magnetic resonance images with a computer system;
(b) accessing motion parameters with the computer system;
(c) generating motion-simulated k-space data with the computer system using a forward model to convert the magnetic resonance images to k-space data while using the motion parameters to apply different degrees of motion to the k-space data;
(d) assembling, by the computer system, a training dataset from the motion-simulated k-space data;
(e) training a neural network on the training dataset using the computer system; and
(f) storing the trained neural network with the computer system.

2. The method of claim 1, wherein the motion parameters comprise three-dimensional motion parameters.

3. The method of claim 2, wherein the motion parameters comprise both three-dimensional translations and three-dimensional rotations.

4. The method of claim 1, comprising accessing pulse sequence data indicating a k-space sampling pattern, and wherein generating the motion-simulated k-space data includes inputting the pulse sequence data to the forward model such that the magnetic resonance images are resampled to the k-space sampling pattern.

5. The method of claim 4, wherein the pulse sequence data indicate a segment ordering for phase-encoding lines for two-dimensional slices in a multislice acquisition.

6. The method of claim 1, wherein assembling the training dataset includes processing the motion-simulated k-space data to extract features indicative of motion artifacts and storing the extracted featured in the training dataset.

7. The method of claim 6, wherein processing the motion-simulated k-space data to extract features indicative of motion artifacts comprises computing a cross-correlation between adjacent phase-encoding lines in the motion-simulated k-space data.

8. The method of claim 7, wherein the extracted features are labeled with different severities of motion artifact based on a magnitude of the cross-correlation.

9. The method of claim 8, wherein the extracted features are labeled with different severities of motion artifact based on the magnitude of the cross-correlation in a central region of the k-space.

10. The method of claim 1, comprising accessing coil sensitivity maps and inputting the coil sensitivity maps as an additional input to the forward model.

11. The method of claim 1, wherein the neural network is a convolutional neural network.

12. The method of claim 11, wherein the convolutional neural network comprises a ResNet architecture.

13. The method of claim 1, wherein the neural network has a plurality of outputs, wherein each of the plurality of outputs corresponds to a different classification of motion artifact severity.

14. The method of claim 1, wherein the motion parameters comprise non-rigid motion parameters.

15. A method for detecting motion artifacts in k-space data acquired with a magnetic resonance imaging (MRI) system, the method comprising:
(a) acquiring k-space data from a subject using the MRI system;
(b) accessing a machine learning model with a computer system, wherein the machine learning model has been trained on training data to detect motion artifacts in k-space data;
(c) inputting the k-space data to the machine learning model, generating motion artifact classification data as an output, wherein the motion artifact classification data indicate a presence and severity of motion artifacts in the k-space data; and
(d) analyzing the motion artifact classification data with the computer system to control operation of the MRI system.

16. The method of claim 15, wherein step (d) includes displaying an alert to a user when motion artifacts are detected above a threshold value of severity based on the analyzing of the motion artifact classification data.

17. The method of claim 16, wherein step (d) includes controlling operation of the MRI system by pausing scanning of the subject when motion artifacts are detected above a threshold value of severity based on the analyzing of the motion artifact classification data.

18. The method of claim 15, wherein the machine learning model is a neural network.

19. The method of claim 18, wherein the neural network comprises a ResNet architecture.
